(19) United States
(12) Patent Application Publication
(10) Pub. No.: US 2004/0225221 A1
(43) Pub. Date: Nov. 11, 2004

(54) Diagnostic ultrasound imaging system with adaptive persistence

(76) Inventor: Lars Jonas Olsson, Woodinville, WA (US)

Correspondence Address: ATL ULTRASOUND, P.O. BOX 3003, 22100 BOTHELL EVERETT HIGHWAY, BOTHELL, WA, 98041-3003, US

(21) Appl. No.: 10/816,331

(22) Filed: Apr. 1, 2004

Related U.S. Application Data
(60) Provisional application No. 60/468,600, filed on May 6, 2003.

(51) Int. Cl. A61B 8/06

(52) U.S. Cl. 600/447

(57) ABSTRACT

A diagnostic ultrasound imaging system includes an ultrasound scanhead coupled to a transmitter and a beamformer. An output of the beamformer is coupled to a persistence processor that determines the extent to which at least one portion of a plurality of image frames vary from one image frame to another. The persistence processor then combines a plurality of the image frames to provide a composite image frame. The number of image frames that are combined to determine the persistence of the composite image and/or the weighting by which they are combined is a function of the extent to which all or a portion of the image frames vary. The composite image is then displayed on a video display.

### TECHNICAL FIELD

This application claims the benefit of Provisional U.S. Patent Application Ser. No. 60/468,600, filed May 6, 2003.

The invention relates diagnostic ultrasound imaging systems, and, more particularly, to a diagnostic ultrasound imaging system that adapts itself to optimally display an image by adjusting the persistence of all or a portion of the image.

### BACKGROUND OF THE INVENTION

The quality of an image obtained with a diagnostic ultrasound imaging system is a function of several imaging parameters. One of these imaging parameters is the persistence at which the image is displayed. Persistence can be thought of as weighted averaging of the displayed image over time or over a number of image frames. A high persistence is obtained by weighted averaging of the displayed image over a longer period of time or over a greater number of imager frames. A low persistence is obtained by weighted averaging the displayed image over a shorter period of time or over a greater number of image frames. Persistence is generally implemented with a temporal filter, linear or nonlinear with finite or infinite impulse response, which is designed to reduce temporal noise in the image.

Increasing the persistence at which an ultrasound image is displayed can, under some circumstances, make it significantly easier to visualize features in the image. For example, the ultrasound reflections from an internal body structure having a small size may be very weak, thus causing the imaging of the structure to be very faint. Averaging the image over several image frames by increasing the persistence of the image has the effect of greatly increasing the signal to noise ratio of the image, thus making it easier to visualize it. However, increasing the persistence at which an image is displayed can be ineffective or counterproductive in some circumstances. For example, if a structure of interest is moving rapidly, averaging the image of the structure over several image frames may not improve the image quality. Instead, averaging the image over several image frames is likely to blur the moving structure. In this regard, movement of an imaging probe or imaged body structures itself do not present a problem because substantially the same images in different locations can be registered with each other by conventional electronic means. However, registering is often impractical if the image itself has changed from one image frame to the next. For example, if the shape of the imaged body structure changes from one frame to the next, it may be impractical to register at least that portion of the image. If the persistence of the displayed image is increased by averaging multiple image frames under those circumstances, the imaged body structure is likely to be blurred.

As a result of these variations in the degree to which adjusting persistence affects an image, it can be very difficult to manually adjust the persistence of a displayed ultrasound image for optimum viewing. Even if it was possible to adjust the persistence of one area of an image for optimum viewing, doing so may cause the persistence of other areas of the image to be adjusted so that features cannot be easily and clearly visualized.

There is therefore a need for a system and method for easily and quickly adjusting the persistence of an ultrasound image for optimum viewing, and doing so in a manner that does not degrade the quality of the image in areas of the image.

### SUMMARY OF THE INVENTION

A method and system for displaying an ultrasound image includes a scanhead for obtaining a plurality of component image frames of body tissue or fluids. The system and method then determines the extent to which at least one portion of each component image frame varies from image frame-to-image frame. A plurality of the component image frames are then combined to provide a composite image frame. The number of component image frames and/or their weighting that are combined to form either one area of the composite image frame or the entire composite image is a function of the determined extent to which at least one portion of each component image frame varies.

### BRIEF DESCRIPTION OF THE DRAWINGS

FIG. 1 is a block diagram of a diagnostic ultrasound imaging system having adaptive persistence capabilities according to of one embodiment of the invention.

FIG. 2 is a block diagram a one embodiment of a persistence processor used in the imaging system of FIG. 1.

FIG. 3 is a block diagram a one embodiment of a frame misregistration determining system that may be used in the imaging system of FIG. 1.

FIGS. 4A and 4B are screen shots showing ultrasound images obtained using the imaging system of FIG. 1 with the adaptive persistence mode disabled and enabled, respectively.

FIG. 5 is a screen shot showing an ultrasound image obtained using the imaging system of FIG. 1 using a different mode of adaptive persistence.

### DETAILED DESCRIPTION OF THE INVENTION

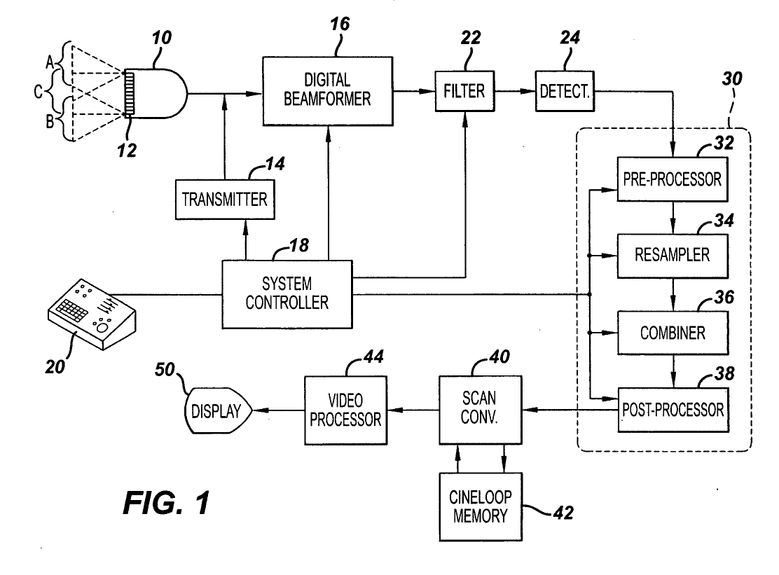

FIG. 1 is a block diagram of one embodiment of an ultrasound imaging system in accordance with the present invention. The ultrasonic diagnostic imaging system includes a scanhead 10. The scanhead 10 includes an array transducer 12 that transmits beams at different angles over an image field denoted by the dashed rectangle and parallelograms. Three groups of scanlines are indicated in FIG. 1, labeled A, B, and C, with each group being steered at a different angle relative to the scanhead 10. The transmission of the beams is controlled by a transmitter 14, which controls the phasing and time of actuation of each element of the array transducer 12 so as to transmit each beam from a predetermined origin along the array transducer 12 and at a predetermined angle.

The echoes returned from along each scanline are received by the elements of the array transducer 12, digitized as by analog to digital conversion, and coupled to a digital beamformer 16. The digital beamformer 16 delays and sums the echoes from the transducer array 12 to form a sequence of focused, coherent digital echo samples along each scanline. The transmitter 14 and beamformer 16 are operated under control of a system controller 18, which in turn is responsive to the settings of controls on a user interface 20 operated by the user of the ultrasound system. As explained below, the user interface 20 includes controls for adjusting the persistence of a display ultrasound image, as well as controls for enabling and controlling various automated persistence adjusting or selecting modes of the ultrasound imaging system. The system controller 18 controls the transmitter 14 to transmit the desired number of scanline groups at the desired angles, transmit energies and frequencies. The system controller 18 also controls the digital beamformer 16 to properly delay and combine the received echo signals for the apertures and image depths used.

The scanline echo signals from the digital beamformer 16 are filtered by a programmable digital filter 22, which defines the band of frequencies of interest. When imaging harmonic contrast agents or performing tissue harmonic imaging, the passband of the filter 22 is set to pass harmonics of the transmit band. The filtered signals are then detected by a detector 24. In a preferred embodiment, the filter 22 and detector 24 include multiple filters and detectors so that the received signals may be separated into multiple passbands, individually detected and recombined to reduce image speckle by frequency compounding. For B mode imaging, the detector 24 performs amplitude detection of the echo signal envelope. For Doppler imaging, ensembles of echoes are assembled for each point in the image and are Doppler processed to estimate the Doppler shift or Doppler power intensity.

The digital echo signals from the detector 24 are processed by a persistence processor 30. In one embodiment, the persistence processor 30 includes a preprocessor 32 that can preweight the signal samples if desired with a weighting factor. For example, signals corresponding to different areas of an image can be given greater persistence. The preprocessor 32 can also preweight different image frames with a weighting factor that is a function of the number of component frames used to form a particular image or the age of the image frames that are combined to form a composite image, as described below. The preprocessor 32 can also weight image frames in other manners. The signal samples from the preprocessor 32 may then undergo a resampling in a resampler 34. The resampler 34 can spatially realign the estimates of one component frame or to the pixels of the display space. After resampling, multiple image frames are added together by a combiner 36. Combining may comprise summation, averaging, peak detection, or other combinational means. The samples being combined may also be weighted by the preprocessor 32 prior to combining in this step of the process, as explained above. Finally, post-processing is performed by a post-processor 38. The post-processor 38 normalizes the combined values to a display range of values. Post-processing can be most easily implemented by look-up tables, although other techniques may be used. The post-processor 38 can simultaneously perform compression and mapping of the range of values for combined image frames to a range of values suitable for display of the ultrasound image.

The combining process explained above may be performed in estimate data space or in display pixel space. In a preferred embodiment, scan conversion is done following the compounding process by a scan converter 40. In other embodiments the scan converter 40 may precede the persistence processor 30 and persistence processing is performed on scan converted image data. The signals corresponding to the image frames may be stored in a Cineloop memory 42 in either estimate or display pixel form. If stored in estimate form, the image frames may be scan converted when replayed from the Cineloop memory 42 for display. The scan converter 40 and Cineloop memory 42 may also be used to render three-dimensional presentations of the spatially compounded images as described in U.S. Pat. Nos. 5,485,842 and 5,860,924, which are incorporated herein by reference. Following conversion by the scan converter 40, the images are processed for display by a video processor 44 and displayed on an image display 50.

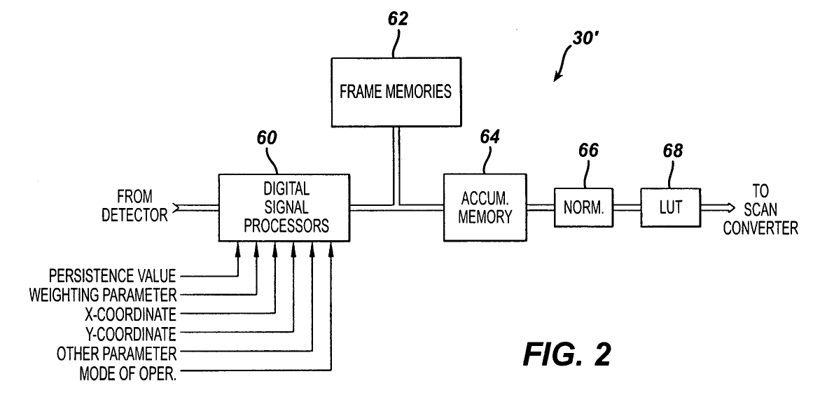

FIG. 2 illustrates an alternative embodiment of a persistence processor 30′ that can be used in the imaging system of FIG. 1. The persistence processor 30′ is preferably implemented by one or more digital signal processors 60, which process the image data in various ways. The digital signal processors 60 can weight the received image data and can resample the image data to spatially align pixels from frame to frame, for instance. The digital signal processors 60 direct the processed image frames to a plurality of frame memories 62 which buffer the individual image frames. The number of image frames capable of being stored by the frame memories 62 is preferably at least equal to the maximum number of image frames to be averaged at maximum persistence. In one embodiment, the frame memories 62 can store sixteen image frames.

In accordance with one aspect of the present invention, the digital signal processors 60 are responsive to persistence control parameters including a persistence value for the entire image or persistence values for respective regions of the image, weighting parameters, operating mode each of which alters the manner in which persistence is selected, and coordinates of an area of interest in a displayed image. Some or all of these parameters can be adjusted using the user interface 20. The digital signal processors 60 select component image frames stored in the frame memories 62 for assembly as a composite image frame in accumulator memory 64. The composite image frame formed in the accumulator memory 64 may be weighted or mapped by a normalization circuit 66, then compressed to the desired number of display bits and, if desired, remapped by a lookup table (LUT) 68. The fully processed composite image frame is then transmitted to the scan converter 40 (FIG. 1) for formatting and display.

As mentioned above, increasing the persistence, i.e., the number of image frames averaged, can generally make it easier to visualize features in an ultrasound image. However, as also previously explained, increasing persistence does not produce any beneficial effect, and may even be detrimental, where the shapes of the structures being imaged are changing rapidly. As also previously explained, the movement that prevents an image from being clearly viewed despite any increase in persistence is movement that changes the shape of the image. Movement of either tissues or the scanhead 10 that does not change the shape of the image does not present a problem because, as previously explained, the resampler 34 can spatially realign the same features in different component image frames.

In accordance with one aspect of the invention, the persistence of all or a portion of the image shown in the display 50 is automatically adjusted based on the rate in which all or a portion of the image changes from frame-to-frame. For this purpose, misregistration of features in a series of component image frames can be measured by a number of conventional or hereinafter developed motion tracking or misregistration measurement methods, such as a correlation block search as described in U.S. Pat. No. 5,782,766, or Doppler tissue velocity sensors as described in U.S. Pat. No. 5,127,409, both of which are incorporated herein by reference. However, these methods generally require large amounts of computation. The effect of misregistration can be detected more simply, without explicit measurement of the motion, by comparing the similarity or difference of one component image frame with subsequent component image frames in the temporal sequence. Similarity or difference metrics, such as cross correlation, normalized cross correlation, or sum of absolute differences (SAD), generally require less computation than motion estimation, and can be used to quantify frame-to-frame similarity or difference in at least one region of interest (ROI) within the image frame. If the sum of absolute differences is zero or very small, then little or no misregistration has occurred between component image frames. Conversely, if the SAD is large, then the misregistration is significant. Therefore, the temporal sequence of the SAD values corresponding to areas in a sequence of image frames gives a running indication of the amount of misregistration due to scanhead motion or tissue movement, and can be used to change the persistence at which all or a portion of an image is displayed.

Misregistration can be measured before or after motion compensation. If it is measured after motion compensation, the residual misregistration after motion compensation can be attributed to tissue motion, out-of-plane motion, tissue compression, and the like. The misregistration measurement can be computed by the resampler 34 if desired in a constructed embodiment.

As mentioned above, in one operating mode, the persistence of all or a portion of the image shown in the display 50 is automatically adjusted. This process can be accomplished through the user interface 20 by dividing a viewing screen of the display 50 into one or more image areas. The digital signal processors 60 then determine the degree of misregistration or residual misregistration in each of these image areas from one component image frame to the next. Based on the degree of misregistration in each of these image areas, the respective image areas from several component image frames are combined to form a respective composite image area. All of the composite image areas are then combined to form the final composite image frame that is used to generate an image shown on the display 50. The number of component image areas combined to create each composite image area and/or the parameters or coefficients used to combine them may vary from image area-to-image area. For example, an image area in which imaged structures are rapidly changing may be displayed in the composite image area using relatively few component image areas or sharply declining weighting of older image areas in the sequence. An image area in which imaged structures are fairly stationary may be displayed using a larger number of component image areas and/or with older component image areas being significantly weighted.

Another mode of operation selectable from the user interface 20 allows a sonographer to select a region of interest in a displayed image. The digital signal processors 60 then adjust the persistence of the entire displayed image based on the degree of image frame-to-image frame misregistration or decorrelation in the selected region of interest. For a relatively low degree of misregistration or decorrelation, a relatively large number of component image frames are averaged to provide a composite image frame that is used to generate an image shown on the display 50. Conversely, for a relatively high degree of misregistration or decorrelation, relatively few component image frames are averaged to provide a composite image frame that is used to generate an image. Other techniques for sensing image frame-to-image frame misregistration or decorrelation may also be used. Some of these other techniques are described in U.S. Pat. No. 6,126,598, which is incorporated herein by reference.

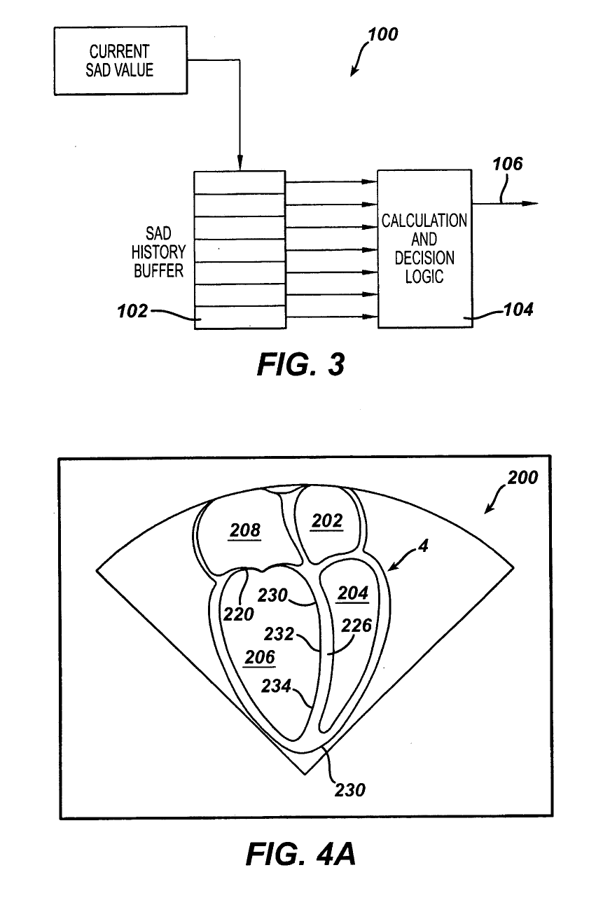

FIG. 3 shows a frame misregistration determining system 100 that may be included in the digital signal processors 60 (FIG. 2). The frame misregistration determining system 100 may be used to compute the SAD values used to adjust the persistence of the displayed image by adjusting the number of component image frames combined to make up all or a portion of the displayed image or the manner in which they are combined. Each time a new SAD metric is calculated, it is stored in a SAD history buffer 102, which retains recently calculated SAD values. When the SAD value drops below a given threshold for a prescribed number of image frames, which levels may be preset or set by the user, a calculation and decision logic unit 104 recognizes the new condition of improved image registration and outputs a signal on line 106 to increase the number of component image frames that are combined to create the displayed image. This signal from the calculation and decision logic unit 104 is used elsewhere in the digital signal processors 60 (FIG. 2) to cause the number of component image frames determined by the calculation and decision logic unit 104 to be combined to generate the composite image frame. A progressive decrease in the SAD value could result in a progressive increase in the number of component image frames combined to form the displayed image. Conversely, a progressive increase in the SAD value could result in a progressive decrease in the number of component image frames combined to form the displayed image. If desired, more recently acquired SAD values stored in the SAD history buffer 102 could be weighted to a greater extent than older SAD values in making the determination whether to increase or decrease the persistence of the displayed image.

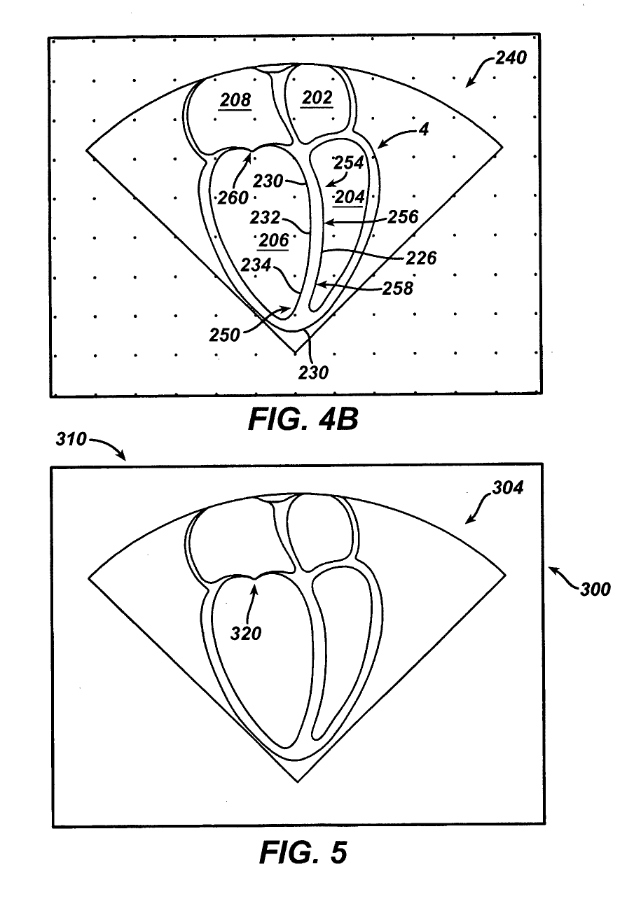

An example of the operation of the diagnostic ultrasound imaging system of FIG. 1 is shown in FIGS. 4A and 4B. A B-mode ultrasound image 200 of a heart H is shown in FIG. 4A. The image 200 shows a cross-section of the heart H showing 4 chambers 202, 204, 206, 208, two of which 206, 208 are connected to each other by a mitral valve 220. As is well known in the field of physiology, the position of the mitral valve 220 changes as the heart H operates to pump blood. As also shown in FIG. 4A, the chambers 202, 204 are separated from each other by a wall 226, which is relatively stationary. However, because of particular conditions under which the ultrasound image was taken, portions of the wall 226 do not reflect ultrasound echoes as well as other areas of the heart H. As a result, the image drops out in areas 230, 232, 234 of the wall 226 so that the wall 226 cannot be visualized well in these areas. Even more stationary than the wall 226 is the apex 230 of the heart H.

The ultrasound image 200 shown in FIG. 4A can be more easily viewed by using the imaging system of FIG. 1 to adaptively adjust the persistence of an image 240 displayed on a screen 244 of the display 50, which is shown in FIG. 4B. Although not displayed with the image 240 in FIG. 4B, the screen 244 is divided into a plurality of image areas shown by the dotted lines in FIG. 4B. The individual composite image frames are likewise divided into the same image areas, and the frame-to-frame misregistration in each of these component image areas is determined as explained above. The imaging system of FIG. 1 then combines these component image areas from several component image frames to provide respective composite image areas that together form the composite image frame used to form the image 240 shown in FIG. 4B. The system displays the image 240 in image area 250 with a high persistence using a relatively large number of component image areas because the image features do not vary significantly from frame-to-frame in this image area 250. The system displays the image 240 in image areas 254, 256, 258 with a moderate persistence using a fewer number of component image areas because, although the image features vary from frame-to-frame in these image areas 254-258, they do not vary too greatly. However, the increased persistence in these image areas 254-258 achieved by using multiple component image areas eliminates drop outs in the areas 230, 232, 234 of the wall 226. As a result, the wall 226 can be visualized significantly better in these areas 230-234 compared to how well they can be visualized in the image 200 shown in FIG. 4A. Finally, the system displays the mitral valve 220 in image area 260 with very little persistence using one or a small number of component image areas because, as mentioned above, the mitral valve 220 moves rapidly as the heart H beats. By adaptively adjusting the persistence in each of the image areas shown in FIG. 4B, the image 240 is displayed with optimum persistence in each area of the image areas.

The operation of the ultrasound imaging system of FIG. 1 in another mode of operation is illustrated in FIG. 5. In this mode of operation, a cursor 300 is displayed along the vertical axis of the screen 244 of the display 50 on which an image 304 is shown, and a similar cursor 310 is displayed along the horizontal axis of the screen 244. The positions of the cursors 300, 310 along their respective axes can be adjusted with the user interface 20 in a conventional manner. The position of the cursor 300 designates an area of the image 304 along the vertical axis, and the position of the cursor 300 designates an area of the image 304 along the horizontal axis. Together, the cursors 300, 310 designate a specific area 320 of the image 304. Other means could also be used to designate the area 320, such as a light pen (not shown), pointing device (not shown), etc. The system of FIG. 1 determines the misregistration of the image from frame-to-frame in this area 320, and adjusts the persistence of the entire image 304 based on that determination.

From the foregoing it will be appreciated that, although specific embodiments of the invention have been described herein for purposes of illustration, various modifications may be made without deviating from the spirit and scope of the invention. For example, although two operating modes for the ultrasound imaging system of FIG. 1 has been specifically shown in FIGS. 4 and 5, it will be understood that other modes may be used to adaptively adjust the persistence of a displayed image. Accordingly, the invention is not limited except as by the appended claims.

1. A method of displaying an ultrasound image, comprising:
obtaining a plurality of component image frames of body tissue or fluids;
determining the extent to which at least one portion of each component image frame varies from image frame-to-image frame;
combining a plurality of the component image frames to provide a composite image frame, the number and/or weighting of component image frames that are combined in at least one area of the composite image frame being a function of the determined extent to which at least one portion of each component image frame varies; and
displaying an image corresponding to the composite image frame.

2. The method of claim 1 wherein the act of determining the extent to which at least one portion of each component image frame varies from image frame-to-image frame comprises determining the extent to which a single portion of each component image frame varies from image frame-to-image frame.

3. The method of claim 2, further comprising manually designating the single portion of each component image frame in which the determination is made of the extent to which the single portion of each component image frame varies.

4. The method of claim 3 wherein the act of manually designating the single portion of each component image frame comprises designating the single portion on the displayed image.

5. The method of claim 1 wherein the act of determining the extent to which at least one portion of each component image frame varies from image frame-to-image frame comprises determining the extent to which each of a plurality of portions of each component image frame varies from image frame-to-image frame.

6. The method of claim 1 wherein the act of combining a plurality of the component image frames to provide a composite image frame comprises weighting the contribution that each of the component image frames makes to the composite image frame so that different component image frames contribute to the composite image frame in differing degrees.

7. The method of claim 6 wherein the act of weighting the contribution that each of the component image frames makes to the composite image frame comprises weighting the contribution that each of the component image frame makes based on the lapse in time since the component image frame was obtained.

8. The method of claim 6 wherein the act of weighting the contribution that each of the component image frames makes to the composite image frame comprises weighting the contribution that each of the component image frames makes based on the number of component image frames combined to provide the composite image frame.

9. A method of displaying an ultrasound image, comprising:
obtaining a plurality of component image frames of body tissues or fluids;
dividing each component image frame into a plurality of image areas, each of the image areas in a component image frame representing substantially the same portion of the body tissues or fluids that is represented by a corresponding image area of the other component image frames;
determining the extent to which corresponding image areas of the plurality of component image frames vary from image frame-to-image frame;
combining the corresponding image areas in each of the plurality of the component image frames to provide respective composite image areas in a composite image frame, the number and/or weighting of image areas that are combined to form each of the composite image areas being a function of the determined extent to which the respective corresponding image areas vary; and
displaying an image corresponding to the composite image frame.

10. The method of claim 9 wherein the act of combining the corresponding image areas in each of the plurality of the component image frames to provide respective composite image areas comprises weighting the contribution that each image area in each component image makes to the respective composite image area so that corresponding image areas from different component image frames contribute to the respective composite image area in differing degrees.

11. The method of claim 10 wherein the act of weighting the contribution that each of the image areas in each component image frame makes to the respective composite image area comprises weighting the contribution that each of the image areas in each component image frame makes based on the lapse since the component image frame was obtained.

12. The method of claim 10 wherein the act of weighting the contribution that each of the image areas in each component image frame makes to the respective composite image area comprises weighting the contribution that each of the image areas in each component image frame makes based on the number of component image areas that are combined to form the respective composite image area.

13. An diagnostic ultrasound imaging system, comprising:
an ultrasound scanhead (10) having a plurality of transducer elements;
a transmitter (14) coupled to the scanhead (10), the scanhead (10) being operable to apply a transmit signal to the scanhead (10);
a beamformer (16) coupled to the scanhead (10), the beamformer (16) being operable to receive signals corresponding to ultrasound echoes from the scanhead (10) and generate a plurality of component image frames corresponding thereto;
a persistence processor (30) coupled to the beamformer (16) to receive signals corresponding to each of a plurality of the component image frames, the persistence processor (30) being operable to determine the extent to which at least one portion of a plurality of the component image frames vary from one image frame to another image frame, the persistence processor (30) further being operable to combine a plurality of the component image frames to provide a composite image frame, the number and/or weighting of component image frames that are combined by the persistence processor (30) in at least one area of the composite image frame being a function of the determined extent to which at least one portion of a plurality of the component image frames vary;
a video processor (44) coupled to the persistence processor (30), the video processor (44) receiving signals corresponding to the composite image frame and generating from the signals corresponding to the composite image frame corresponding video signals; and
a display (50) coupled to the video processor (44) for receiving the video signal and displaying a corresponding ultrasound image.

14. The diagnostic ultrasound imaging system of claim 13 wherein the persistence processor (30) comprises:
a pre-processor (32) coupled to the beamformer (16), the pre-processor (32) being operable to preweight the signals corresponding to each of a plurality of the component image frames;
a resampler (34) coupled to the preprocessor (32), the resampler (34) being operable to process signals from the preprocessor (34) to spatially realign the component image frames;
a combiner (36) coupled to the resampler (34), the combiner (36) being operable to combine a plurality of the component image frames to provide the composite image frame; and
a post-processor (38) coupled to the combiner (36), the post-processor (38) being operable to normalize signals corresponding to the composite image frame.

15. The diagnostic ultrasound imaging system of claim 14 wherein the pre-processor (32) is operable to preweight the signals corresponding to each of a plurality of the component image frames with a weighting factor that is a function of the number of component image frames that are combined to form the composite image frame.

16. The diagnostic ultrasound imaging system of claim 14 wherein the pre-processor (32) is operable to preweight the signals corresponding to each of a plurality of the component image frames by the age of the component image frames that are combined to form the composite image.

17. The diagnostic ultrasound imaging system of claim 13 wherein the diagnostic ultrasound imaging system further comprises a user interface (20), and wherein the persistence processor (30) comprises:
a digital signal processor (60) coupled to the beamformer (16) and to the user interface, the digital signal processor (60) being operable to receive from the user interface (20) a plurality of processing parameters and to process the signals corresponding to each of a plurality of the component image frames based on the processing parameters;
an image frame memory (62) coupled to receive and store the signals corresponding to each of a plurality of the component image frames; and
an accumulator memory (64) coupled to the digital signal processor (60) and to the image frame memory (62), the accumulator memory (64) being operable to receive from the image frame memory (62) signals corresponding a plurality of the component image frames selected by the digital processor (60) and to store the signals for coupling to the video processor (44).

18. The diagnostic ultrasound imaging system of claim 17, wherein the digital signal processor comprises a frame misregistration system, the frame misregistration system comprising:
a history buffer (102) receiving and storing data indicative of the extent to which at least one portion of the plurality of component image frames vary from one image frame to the next; and
a calculation and decision logic unit (104) coupled to the history buffer (102) to receive the data stored in the history buffer (102), the calculation and decision logic unit (104) being operable to determine based on the data stored in the history buffer (102) the number of component image frames that should be combined to generate the composite image frame.

19. The diagnostic ultrasound imaging system of claim 13 wherein the persistence processor (30) is operable to determine the extent to which at least one portion of a plurality of the component image frames vary from one image frame to another by determining the extent to which a single portion of each component image frame varies from one image frame to another image frame.

20. The diagnostic ultrasound imaging system of claim 19 wherein the diagnostic ultrasound imaging system further comprises a user interface (20), and wherein the persistence processor (30) is operable to determine the extent to which a single portion of the component image frames varies by manually designating the single portion with the user the user interface (30).

21. The diagnostic ultrasound imaging system of claim 13 wherein the persistence processor (30) is operable to determine the extent to which at least one portion of a plurality of the component image frames vary from one image frame to another by determining extent to which each of a plurality of portions of each component image frame varies from image frame to another image frame.

22. The diagnostic ultrasound imaging system of claim 13 wherein the persistence processor (30) is operable to weight the contribution that each of the component image frames makes to the composite image frame so that different component image frames contribute to the composite image frame in differing degrees.
